(19) United States
(12) Patent Application Publication
(10) Pub. No.: US 2023/0301535 A1
(43) Pub. Date: Sep. 28, 2023

(54) Artificial Intelligent Stent with Endovascular Nano-structured Flowmeter Layer for Monitoring Mental Performance, Re-stenosis and Thromboembolism

(71) Applicants: Philip Chidi Njemanze, Owerri (NG); Michelle Nkem Chidi-Njemanze, Owerri (NG); Philip Chidi Njemanze, JR., Owerri (NG); Nicole Odera Chidi-Njemanze, Owerri (NG)

(72) Inventors: Philip Chidi Njemanze, Owerri (NG); Michelle Nkem Chidi-Njemanze, Owerri (NG); Philip Chidi Njemanze, JR., Owerri (NG); Nicole Odera Chidi-Njemanze, Owerri (NG)

(21) Appl. No.: 17/654,596

(22) Filed: Mar. 14, 2022

(51) Int. Cl. A61B 5/026; A61B 5/16; A61B 5/00; A61F 2/70; A61B 5/18; G16H 50/20

(52) U.S. Cl. CPC A61B 5/026; A61B 5/165; A61B 5/18; A61B 5/4088; A61B 5/4806; A61B 5/686; A61F 2/70; G16H 50/20

(57) ABSTRACT

This invention is related to artificial intelligent (AI) system with biodegradable endovascular nanoscale-structured flowmeter layer for monitoring cerebral blood flow during mental performance, blood flow through in-stent re-stenosis (ISR) and thromboembolism. The invention is based on blood flow inducing changes in magnetization of nanoscale microwave ferrites. These changes in magnetization, then interact with the microwave in a frequency-dependent manner using a microprocessor for processing and transmission via cellular phone network to human-machine interface for control of computers, machines or weapon systems. It detects reduction of blood flow through ISR and microembolic signals due to thromboembolism of the vessel much earlier before severe symptoms develop.

### CROSS-REFERENCE TO RELATED APPLICATION

U.S. Patent Documents

| Document Number | | Date | Name | Cited |
|---|---|---|---|---|
| 1. | 6468219B1 | 10-2002 | Njemanze | Inventor |
| 2. | 6390979 B1 | 05-2002 | Njemanze | Inventor |
| 3. | 6663571 | 16-2003 | Njemanze | Inventor |
| 4. | 100770606 | 04-2002 | Njemanze | Inventor |
| 5. | 5295491 | 02-1997 | Quintana-Almagro et al. | Inventor |
| 6. | 5724987 | 03-1998 | Gevins et al. | Inventor |
| 7. | 5295491 | 03-1994 | Gevins | Inventor |
| 8. | 5771261 | 06-1998 | Anbar | Inventor |
| 9. | 6126595 | 10-2000 | Amano et al. | Inventor |
| 10. | 6258032 | 07-2001 | Hammesfahr | Inventor |
| 11. | 11208720 | 12-2021 | Junkar et al. | Inventor |
| 12. | 11207448 | 12-2021 | Sasaki et al. | Inventor |

### OTHER PUBLICATIONS

Albiero R, Nishida T, Adamian M. et al. Edge restenosis after implantation of high activity P radioactive β-emitting stents. Circulation, 2000;101:2454-2457.

Alfonso F. Treatment of drug-eluting stent restenosis the new pilgrimage: Quo vadis? J Am Coll Cardiol, 2010;55:2717-2720

Aoki J, Serruys PW, van Beusekom H, et al. Endothelial Progenitor Cell Capture by Stents Coated With Antibody Against CD34: The HEALING-FIM (Healthy Endothelial Accelerated Lining Inhibits Neointimal Growth-First In Man) Registry, J Am Coll Cardiol, 2005;45(10):1574-1579. https://doi.org/10.1016/j.jacc.2005.01.048

Basso A, De Renzi E, Faglioni P, Scotti G, Spinnler H. Neuropsychological evidence for the existence of cerebral areas critical to the performance of intelligence tasks. Brain, 1973; 96:715-728.

Bourantas CV, Onuma Y, Farooq V, et al. Bioresorbable scaffolds: Current knowledge, potentialities and limitations experienced during their first clinical applications. Int J Cardiol, 2012;167:11-21.

Cabeza R, Nyberg L. Neural bases of learning and memory: functional neuroimaging evidence. Curr Opin Neurol 2000;13(4):415-421.

Carpenter PA, Just MA, Shell P. What one intelligence test measures: A theoretical account of the processing in the Raven progressive matrices test. Psychological Review, 1990; 97:404-431.

Clark JM, Skolnick BE, Gelfand R, Farber RE, Stierheim M, Stevens WC, Beck G Jr, Lambertsen CJ. Relationship of 133Xe cerebral blood flow to middle cerebral arterial flow velocity in men at rest. J Cereb Blood Flow Metab. 1996;16:1255-62. doi: 10.1097/00004647-199611000-00021. PMID: 8898699.

Daemen J, Serruys P W. Drug-eluting stent update 2007: Part I. A survey of current and future generation drug-eluting stents: Meaningful advances or more of the same? Circulation, 2007;116:316- 328.

Duncan J, Seitz RJ, Kolodny J, Bor D, Herzog H, Ahmed A, Newell FN, Emslie H. A neural basis for general intelligence. Science, 2000;289:457-460.

Feynman RP, Leighton RB, Sands M. The Feynman Lectures on Physics. Mainly Electromagnetism and Matter. Vol. II. The New Millennium Ed. Basic Books; 2010. p. 566. ISBN: 978-0-465-07998-8

Fletcher P, Büchel C, Josephs O, Friston K, Dolan R. Learning-related neuronal responses in prefrontal cortex studied with functional neuroimaging. Cereb Cortex, 1999;9(2):168-78. doi: 10.1093/cercor/9.2.168. PMID: 10220229.

Hartmann, J. Theory of the Laminar Flow of an Electrically Conductive Liquid in a Homogeneous Magnetic Field. Fys. Med., 1937;15:1-27.

Hong Y, Zhou F, Hua Y, Zhang X, Ni C, Pan D, et al. A strongly adhesive hemostatic hydrogel for the repair of arterial and heart bleeds. Nat Commun. 2019 May 14;10(1):2060. doi: 10.1038/s41467-019-10004-7. PMID: 31089131; PMCID: PMC6517429.

Huai-Wu ZT, Li J, Hua S, Chuan ZT, Yang L, Liang ZZ. Development and application of ferrite materials for low temperature co-fired ceramic technology. Chinese Physics B. 2013;22(11):117504.

Hund-Georgiadis M, von Cramon DY. Motor-learning-related changes in piano players and non-musicians revealed by functional magnetic-resonance signals. Exp Brain Res, 1999;125(4):417-25. doi: 10.1007/s002210050698. PMID: 10323287.

Jackson JD. Classical Electrodynamics. John Wiley & Sons, Inc.; 1962. P. 656.

Jensen AR. The g beyond factor analysis. In A. R. Ronning, J. A. Glover, J. C. Conoley, & J. C. Witt (Eds.). The influence of cognitive psychology on testing (pp. 87-142). Hillsdale, NJ: Erlbaum. 1987.

Jung RE, Haier RJ, Yeo RA, Rowland LM, Petropoulos H, Levine AS, et al. Sex differences in N-acetylaspartate correlates of general intelligence: an 1H-MRS study of normal human brain. Neuroimage, 2005;26:965-972. Epub 2005/06/16. https://doi.org/10.1016/j.neuroimage.2005.02.039 PMID:15955507.

Landau LD, Lifshitz EM. Electrodynamics of Continuous Media. Course of Theoretical Physics. Vol 8. 2nd ed. Pergamon Press; 1984. p. 457.

Leon MB, Teirstein PS, Moses JW, et al. Localized intracoronary gamma-radiation therapy to inhibit the recurrence of restenosis after stenting. N Engl J Med, 2001;344:250-256.

Lorrain P, Corson D. Electromagnetic Fields and Waves. 2^{nd} ed.WH Freeman and Company; 1970. 696 p. ISBN: 0-7167-0331-9

Moravej M, Mantovani D. Biodegradable metals for cardiovascular stent application. Int J Mol Sci, 2011;12:4250-4270.

Morrish AH. The Physical Principles of Magnetism. John Wiley & Sons, Inc.; 1965. p. 680.

Nair LS, Laurencin CT. Biodegradable polymers as biomaterials. Prog Polym Sci, 2007;32:762-798.

Newby AC, Zaltsman AB. Molecular mechanisms in intimal hyperplasia. J Pathol, 2000;190:300-309.

Njemanze PC. Critical limits of pressure-flow relation in the human brain. Stroke 1992;23(12):1743-1747.

Njemanze PC, Antol PJ, Lundgren CE. Perfusion of the visual cortex during pressure breathing at different high-G stress profiles. Aviat Space Environ Med 1993;64(5):396-400.

Njemanze PC. Cerebral circulation dysfunction and hemodynamic abnormalities in syncope during upright tilt test. Can J Cardiol 1993; 9(3):238-242.

Njemanze PC. Cerebral lateralization in random letter task in the visual modality: A transcranial Doppler study. Brain and Language, 1996;63:315-325.

Njemanze PC. Cerebral lateralization for motor tasks in simulated microgravity. A transcranial Doppler technique for astronauts. J Gravit Physiol, 2002;9:33-34.

Njemanze PC. Asymmetry in cerebral blood flow velocity with processing of facial images during head-down rest. Aviat. Space Environ. Med. 2004;75:800-805.

Njemanze PC. Cerebral lateralization and general intelligence: gender differences in a transcranial Doppler study. Brain Lang. 2005;92:234-239. Epub 2005/02/22. https://doi.org/10.1016/j.band1.2004.06.104 PMID: 15721956.

Njemanze PC. Cerebral lateralisation for facial processing: Gender-related cognitive styles determined using Fourier analysis of mean cerebral blood flow velocity in the middle cerebral arteries. Laterality Asymmetries Body, Brain Cogn 2007;12(1):31-49.

Njemanze PC. Asymmetric neuroplasticity of color processing during head down rest: a functional transcranial Doppler spectroscopy study. J Gravit Physiol. 2008;15:49-59.

Njemanze PC. Gender-related asymmetric brain vasomotor response to color stimulation: a functional transcranial Doppler spectroscopy study. Exp Transl Stroke Med.2010;2:21. Epub 2010/12/02. https://doi.org/10.1186/2040-7378-2-21 PMCID: PMC3006356. PMID: 21118547

Njemanze PC. Gender-related differences in physiologic color space: a functional transcranial Doppler (fTCD) study. Exp Transl Stroke Med. 2011;3:1. Epub 2011/Feb/12. https://doi.org/10.1186/2040-7378-3-1 PMCID: PMC3050757. PMID: 21310045

Njemanze PC, Kranz, M, Amend M, Hauser J, Wehrl H, Brust P. Gender differences in cerebral metabolism for color processing in mice: A PET/MRI study. PLoS ONE 2017;12:e0179919.

Njemanze P, Kranz M, Brust P. Fourier Analysis of Cerebral Metabolism of Glucose: Gender Differences in Mechanisms of Color Processing in the Ventral and Dorsal Streams in Mice. Forecasting 2019;1(1):135-156.

Njemanze P, Kranz M, Brust P. Gender Differences in Cerebral Metabolism Induced by Polarized Light in Mice Brain: A Quantum Coherence Model. Int J Nuclear Med Radioactive Subs 2020;3(3):000135.

Ong A T, McFadden E P, Regar E, et al. Late angiographic stent thrombosis (LAST) events with drug-eluting stents. J Am Coll Cardiol, 2005;45:2088-2092.

Özgür U, Alivov Y, Morkoc HA. Microwave ferrite, part 1: Fundamental properties. Journal of Materials Science: Materials in Electronics. 2009;20(9):789-834

Papaioannou T, Stefanadis C. Vascular wall shear stress: basic principles and methods. Hellenic J Cardiol 2005;46(1):9-15.

Pardavi-Horvath M. Microwave applications of soft ferrites. J. Magn. Magn. Mater. 2000;215-216:171-183.

Poldrack, RA., Prabhakaran, V, Seger, CA, & Gabrieli, JDE. Striatal activation during acquisition of a cognitive skill. Neuropsychology, 1999;13(4);564-574. https://doi.org/10.1037/0894-4105.13.4.564

Prabhakaran V, Smith JA, Desmond JE, Glover GH, Gabrieli JD. Neural substrates of fluid reasoning: an fMRI study of neocortical activation during performance of the Raven’s Progressive Matrices Test. Cogn Psychol. 1997;33:43-63.

Ranade S V, Miller K M, Richard R E, et al. Physical characterization of controlled release of paclitaxel from the TAXUS® Express2® drug-eluting stent. J Biomed Mater Res A, 2004;71A:625-634.

Raven JC. Guide to progressive matrices. London: H.K. Lewis. 1938.

Shenhreen A, Manoh K, Veerakumar V, Bijoy KK. Microwave monolithic filter and phase shifther using magnetic nanostructures. American Institute of Physics. 2018;8:056624

Shit GC, Roy M. Effect of induced magnetic field on blood flow through a constricted channel: an analytical approach. J Mech Med Biol. 2016;16(3):1650030 https://doi.org/10.1142/50219519416500305

Silva MB Jr, Hobson RW 2nd, Jamil Z, Araki CT, Goldberg MC, Haser PB, et al. A program of operative angioplasty: endovascular intervention and the vascular surgeon. J Vasc Surg. 1996;24(6):963-971; discussion 971-3. doi: 10.1016/s0741-5214(96)70042-3. PMID: 8976350.

Snow RE, Kyllonen PC, Marshalek B. The topography of ability and learning correlations. In R. J. Sternberg (Ed.), Advances in the psychology of human intelligence. Hillsdale, NJ: Erlbaum. 1984.

Son D, Lee J, Lee DJ, Ghaffari R, Yun S, Kim SJ, et al. Bioresorbable electronic stent integrated with therapeutic nanoparticles for endovascular diseases. ACS Nano. 2015;23;9(6):5937-5946. doi: 10.1021/acsnano.5b00651. Epub 2015 Apr 28. PMID: 25905457.

Spearman CE. General intelligence, objectively determined and measured. Am J Psychol, 1904;15:201-293.

Spearman CE. The nature of intelligence and the principles of cognition. London: Macmillian. 1923.

Spearman CE. The abilities of man. New York: Macmillian. 1927.

Stone G W, Ellis SG, Cox DA, et al. One-year clinical results with the slow-release, polymer-based, paclitaxel-eluting TAXUS stent the TAXUS-IV trial. Circulation, 2004;109:1942-1947.

Tranel D, Damasio H, Denburg NL, Bechara A. Does gender play a role in functional asymmetry of ventromedial prefrontal cortex? Brain. 2005;128:2872-2881. Epub 2005/10/01. https://doi.org/10.1093/brain/awh643 PMID: 16195242.

Tung R, Kaul S, Diamond G A, et al. Narrative review: Drug-eluting stents for the management of restenosis: A critical appraisal of the evidence. Ann Intern Med, 2006;144:913-919.

Welt FGP, Rogers C. Inflammation and restenosis in the stent era. Arterioscler Thromb Vasc Biol, 2002;22:1769-1776.

Wolf K V, Zong Z, Meng J, et al. An investigation of adhesion in drug-eluting stent layers. J Biomed Mater Res A, 2008;87:272-281.

### STATEMENT REGARDING FEDERALLY SPONSORED RESEARCH OR DEVELOPMENT

Not applicable

### REFERENCE TO MICROFICHE APPENDIX

Not Applicable

### BACKGROUND OF THE INVENTION

This invention is related to system for measurement of cerebral blood flow passing through a flow sensor layer of a vascular stent for monitoring mental performance, in-stent re-stenosis and thromboembolism. The stent includes biodegradable endovascular nanoscale-structured flowmeter layer for monitoring mental performance, and blood flow through in-stent re-stenosis (ISR) and thromboembolism. It applies artificial intelligent (AI) hence referred to here as AI-NANOFLOWMETER system. The AI-NANOFLOMETER detects changes in cerebral blood flow during mental performance by inducing changes in frequency of the nanoparticles placed on the surface of a stent or other supporting matrix on the vascular endothelium. Although, other forms of flow sensors could be applied for use in the present invention, we would by way of example describe one embodiment of the present invention based on blood flow inducing changes in magnetization of nanoscale multiple ferro- or ferrimagnetic microwave ferrites. These changes in magnetization, then interact with the microwave in a frequency-dependent manner and processed by a microprocessor to communicate including using 5G cellular phone network with human-machine interface for control of the machines or weapon systems. Similarly, reduction of blood flow through ISR or detection of microembolic signals in a case of thromboembolism of the coronary artery or leg vascular stent, could be diagnosed early before severe symptoms develop.

In recent years, there has been effort towards development of human-machine interface systems for mind control of machines, robots and weapon systems. Studies using modern imaging techniques in cognitive neuroscience have demonstrated precise monitoring of mental performance [Njemanze, 2005; Tranel, 2005]. However, monitoring mental performance for applications of mind-control of machines has not been an easy task. Current approaches have attempted using tools like genetic engineering of the human brain, nanotechnology and infrared beams. There is no comprehensive and universal approach to monitoring of mental performance for human-machine interface.

The basic mechanism of mental performance is still subject to much controversy. Much of the discussion have centered on mechanisms of general intelligence. The focus on intelligence is important, since the binary choice that denotes consent or no-consent must be made intelligently. Therefore, the objective is to monitor that the decision was made by the brain hemisphere responsible for intelligent decision-making. The system must detect that the input was from the hemisphere where the center of intelligence is located, while at the same time the contra-lateral hemisphere has minimal input. Therefore, the debate on the location of human intelligence is very relevant to the discussion of mind-control of machines.

The theory of general intelligence or g-factor that contributes to success in diverse forms of cognitive activity was postulated over a century ago [Spearman, 1904, 1923, 1927; Jensen, 1987]. It was proposed that, the g-factor could be tested using psychometric tests at the center of space [Snow et al., 1984]. Psychometric tests including Raven’s test [Raven, 1938] and other complex reasoning tests were placed at the center, while simpler tests were placed toward the periphery. This may suggest that, psychometric tests such as Raven’s test could provide a good measure of general intelligence and should account for a good deal of the reasoning in other tests in the center of space [Carpenter et al., 1990]. The g-factor otherwise described as “general intelligence” refers to a construct underlying a small range of tests, namely those at the center of space.

There are neural anatomic networks for processing of tasks of general intelligence. During Raven’s Progressive Matrices (RPM) tasks, there is the necessity of keeping several conceptual formulations in mind, in other words, requiring a working memory function [Carpenter et al., 1990] involving the prefrontal cortex [Prabhakaran et al., 1997]. It has been suggested that, post-rolandic structures may be more critical for this task as shown in patients with brain lesions [Basso et al., 1973]. Duncan et al. [2000] demonstrated in normals using positron emission tomography (PET) studies, that high g tasks do not show diffuse recruitment of multiple brain regions, instead they are associated with selective recruitment of lateral prefrontal cortex in one or both hemispheres. Njemanze [2005] demonstrated that successful resolution of RPM was associated with comparatively greater increase in cerebral blood flow velocity in the right middle cerebral artery (RMCA) in men but in the left middle cerebral artery (LMCA) in women indexed by transcranial Doppler ultrasound. It was concluded that, general intelligence was located in the right hemisphere in men, but in the left hemisphere in women. The latter has been confirmed with studies using positron emission tomography and magnetic resonance spectroscopy [Tranel et al. 2005; Jung et al. 2005].

The presentation of visual information in colors could be used to stimulate brain responses. It has been demonstrated using cerebral blood flow velocity indexed by transcranial Doppler ultrasound that, the right visual cortex processes colors in men, while the left visual cortex processes colors in women [Njemanze, 2008, 2010, 2011]. The opposite trend was observed in animal experiments using magnetic resonance imaging (MRI) and PET, which demonstrated that color stimulation evoked cerebral blood flow increase the right visual cortex in female mice but in the left visual cortex in male mice [Njemanze et al., 2017; 2019; 2020].

Similarly, motor skill learning is associated with the activation of motor areas of the frontal lobes [Cabeza and Nyberg, 2000], notably the premotor and supplementary motor cortex (lateral and medial Broadman Area 6), and also parietal areas. This latter is associated with changes in cerebral blood flow. These regions of the brain receive blood supply predominantly from the middle cerebral arteries (MCA). Recently, studies using functional magnetic resonance imaging (fMRI) have examined motor skill learning of complex finger movements in piano players and non-musicians [Hund-Gerogiadis and von Cramon, 1999]. Njemanze (2002) demonstrated the changes in cerebral blood flow velocity during motor tasks.

Non-motor skill learning using written language could also be used to stimulate specific brain areas. The brain activity during artificial grammar learning evoked changes in specific areas on the brain [Fletcher et al., 1999]. Learning grammar activated left PFC, whereas reduced instance memory attenuated right PFC [Poldrack et al., 1999]. The visual processing of letters by both hemispheres evoked changes in cerebral blood flow velocity documented using the transcranial Doppler technique [Njemanze, 1996].

Furthermore, the processing of faces could be used to stimulate cerebral blood flow responses in the human brain. It has been demonstrated that facial processing stimulated increase in the cerebral blood flow velocity indexed by transcranial Doppler ultrasound in the right hemisphere in men, but in the left hemisphere in women [Njemanze, 2007]. The processing of faces was studied using transcranial Doppler during head-down tilt to simulate the effects of microgravity [Njemanze, 2004].

Several other attempts have been undertaken to test mental performance. Gevins (1994) described a testing method and system for testing the mental performance capability of a human subject, which includes a digital computer workstation for presenting a test to the subject, such as visuomotor memory task in U.S. Pat. No. 5,295,491 1994. During testing, the subject’s physiological variables including brain waves, eye activity, scalp and facial muscle activity, heart activity, respiration and/or skin conductance are analyzed. Givens et al. (1998) described a computer-aided training system that uses electroencephalograms (EEGs) recorded from the trainee’s scalp to alter the training protocol being presented by the computer, for example to present a new task to the trainee when he or she has mastered and automatized the current task in U.S. Pat. No. 5,724,987 1998. The functions are calculated by computer neural networks and consist of a combination of EEG and other physiological variables, which specifically characterize a trainee’s level of focused attention and neurocognitive workload and his “neurocognitive strategy”. The “critical limit of neurocognitive workload” refers to a measurable cutoff point after which error rates on the task appear to rise dramatically in combination with change in neurocognitive strategy. There is need to define related terms of mental performance monitoring.

‘Mental performance’ of a subject (human or animal) refers to a cumulative physiologic brain response in a subject performing tasks of cognitive functions including linguistic, non-linguistic, visual, auditory or psychomotor stimuli. The term natural intelligence refers to mental performance in a human subject accompanied with detectable lateralization changes in cerebral blood flow velocity. The term enhanced natural intelligence systems (eNI) refers to computer programs and operating systems that are interfaced with natural intelligence in a manner that will enhance overall performance of the subject and efficiency of the programs. The term artificial intelligence (AI) refers to computer models of brain function that uses adjustable weighted connections to machine learning and recall. The model when organized in ‘neuronal circuits’ comprising one or several neurons in a network is referred to as neural networks. A ‘hybrid neural network’ is a neural set with crisp signals and weights and crisp transfer function. The term ‘mental signature’ refers to the highly sensitive and specific characteristics of the cerebral blood flow changes and derived laterality indices in response to a particular task presented to a given individual and providing high reproducibility on repeated testing in the same subject. In other words, the term ‘mental signature’ refers to the pattern of changes of neurocognitive strategy in response to tasks in a given subject with high sensitivity, specificity and reproducibility. The neurocognitive strategy used by a subject could describe the subject’s ‘mental state of being’ at any given time. The neurocognitive strategy used for processing a task with the resultant best performance indices is described as the ‘best mental state-of-being’. Having determined the neurocognitive strategy and workload, the proximity to the critical limit will determine the necessity to change “autonomy decision-making level” from an operator to an automated system or mission control. The term “autonomy decision-making level” refers to the level of independence a subject/operator has to take decisions without intervention of a “command-and-control unit” or his ability to override automated tasks on a host computer. The term “command-and-control unit or mission control center” refers to a group of human operators who are involved in definition of mission strategic and tactical objectives, the term is used interchangeable with “mission control”. The invention by Njemanze (2002) described in U.S. Pat. 6,390,979, a non-invasive method and system to determine the mental performance capacity of a human subject based on measurement of cerebral blood flow velocity in cerebral arteries using a transcranial Doppler ultrasound. Cerebral blood flow velocity measurement correlates with cerebral blood flow and metabolism, and hence with mental activity [Clark et al, 1996]. It therefore follows that, monitoring of the cerebral blood flow which varies proportionally with velocity given the constant diameter of the vascular stent could be used to monitor mental performance. Transcranial Doppler ultrasound measures cerebral blood flow velocity in a very small cross-section of the artery called the sample volume, which correlates with mental activity. Similarly, the present invention measures index of cerebral blood flow across a small segment of the brain artery with placement of a vascular stent.

The aforementioned have shown that, different stimuli including intelligence, color, odor, faces, linguistic, non-linguistic, motor and non-motor could be used to cause changes in cerebral blood flow in the brain which could be monitored. The challenge is to provide nano-structuring of the surface of the vascular stent to convert the changes induced by blood flow to frequency signals that interact with microwave for onward transmission to a brain-machine interface system.

Microwaves interact with matter, microscopically, through its constituent atoms, conduction electrons if present, and atomic magnetic dipoles if present. Yet, macroscopically, the effects of microwaves on matter are well described by the four Maxwell equations and the electrodynamic properties of matter: ∈ (electric permittivity), µ (magnetic permeability), and σ (electrical conductivity).

Microwaves interact with different types of matter. The general electrodynamic properties of matter, ∈, µ, and σ, determine completely their behavior when microwaves “hit” them. More specifically, the electric permittivity, ∈, carries information on the polarization of a dielectric specimen (water, vapor, clouds, wood, glass, and so on) and is related to the number of electric dipoles as χ = Nα/((∈₀ - Nαb) and P = (∈ - ∈₀) E, and ∈ = (1 + χ) ∈₀, with P = ∈₀χE and α the molecular polarizability of the medium and is generally anisotropic, i.e., αₓ ≠ α_{y} ≠ α_{z}; hence χ in general is anisotropic and is represented by a tensor in matrix form. The microwaves are absorbed by the electric dipoles which execute damped oscillations at the GHz frequency. The damped motion brings with it a complex ∈ = ∈’ - i∈” which is also a function of frequency [Lorrain and Corson, 1970; Jackson, 1962], in which ∈” takes account of the energy losses. The magnetic permeability, µ, carries information on the magnetization capacity of a material that carries a number N of magnetic dipoles. They are related by µ = µ₀(1 + χₘ), M = χₘH, M = (µᵣ -1)H, and M = ∑mᵢ, where mᵢ are microscopic, atomic magnetic moments (spin, S; orbital, m) [Feynman et al., 2010; Landau and Lifshitz, 1984]. The magnetic dipoles absorb microwave energy because they precess with damping under the torques produced by the microwave’s magnetic field; according to the Landau-Lifshitz equation of motion: M′(t) = γM×H(ω) -αM×(M×H(ω))), in which H(ω) is the magnetic field component of the microwaves, γ is the gyromagnetic ratio, and α is the damping constant [Landau and Lifshitz, 1984]. The precession velocity and hence M′ is different for different H(ω). The losses increase at a higher frequency. The damped precessions bring with them the loss of microwave energy making the magnetic permeability complex frequency-dependent, µ(ω) = µ′(ω) - iµ″(ω). Furthermore, the response of M to H(ω) is almost always direction-dependent, i.e., given H in direction x produces Mₓ, but the same H applied along y or z produces M_{y} ≠ M_{z} ≠ Mₓ, and these responses are properly described with a tensor _{χm}(ω), or tensor µ(ω).

Microwave ferrites are a set of magnetic materials that have been used in multiple microwave applications [Özgür et al., 2009; Pardavi-Hotvath 2000; Shenhreen et al., 2018; Huai-Wu et al., 2013]. Elements such as Fe, Ni, and Co and alloys with other elements (titanium, aluminum) that exhibit relative magnetic permeabilities up to 10 are well known ferromagnetic materials. Within a certain temperature range, ferromagnetic substances have net atomic magnetic moments that line up in such a way that magnetization persists after the removal of the applied field. Below a certain temperature, called the Curie point (or Curie temperature), an increasing magnetic field applied to a ferromagnetic substance will cause increasing magnetization to a high value called the saturation magnetization. This is because a ferromagnetic substance consists of small magnetized regions called domains. The total magnetic moment of a sample of the substance is the vector sum of the magnetic moments of the component domains.

In a situation where the magnetic material is ferro- or ferrimagnetic and it is not magnetically saturated, its magnetic structure is comprised of domains and domain walls; the magnetization, Mₐ, within a domain, a, has a magnitude and a direction, a; the magnetization, M_{b}, within domain b, has another magnitude and another direction, b, and so on. There are walls between the domains which have considerable amount of magnetic energy [Landau and Lifshitz, 1984; Morrish, 1965] and can move in translational or rotating dissipative and anisotropic motion following the LL damped equation of motion given above.

Conventionally, ferri-magnets have low RF loss and are used in passive microwave components such as isolators, circulators, phase shifters, and miniature antennas operating in a wide range of frequencies (1-100 GHz) and as magnetic recording media owing to their novel physical properties. Tuning the frequency of these components has so far been achieved by external magnetic fields provided by a permanent magnet or by passing current through coils. When high frequencies are required, it is plausible to use hexaferrites, such as BaFe₁₂O₁₉ and SrFe₁₂O₁₉, which have high effective internal magnetic anisotropy that also contributes to the permanent bias. Such a self-biased material remains magnetized even after removing the external applied magnetic field, and thus, may not even require an external permanent [Özgür et al., 2009]. On the other hand, in garnet and spinel ferrites, such as Y₃Fe₅O₁₂ (YIG) and MgFe₂O₄, the uniaxial anisotropy is much smaller, and one would need to apply huge magnetic fields to achieve such high frequencies.

When the nanoscale microwave ferrites are placed on the surface of a cylindrical stent in an artery with blood flow, then the magnetic moments change with changing brain perfusion induced by mental performance. For blood flow within a rigid or inelastic, cylindrical and straight vessel, the shear stress is defined by the Haagen-Poisseuille equation:

$$τ = 32 ⋅ μ ⋅ {}_{-} \frac{Q}{π ⋅ {d}^{3}} {}^{\text{or}} τ = 8 ⋅ μ ⋅ \frac{u}{d}$$

where Q is the mean volumetric flow rate, u the mean velocity, and d the vessel diameter. A few assumptions were made to apply in vivo the Haagen-Poisseuille equation, which include the following: (a) the blood is considered as a Newtonian fluid; (b) the vessel cross sectional area is cylindrical; (c) the vessel is straight with inelastic walls; (d) the blood flow is steady and laminar. The Haagen-Poisseuille equation indicates that shear stress is directly proportional to blood flow rate and inversely proportional to vessel diameter [Papaioannou and Stefanadis, 2005]. There is a constant cylindrical cross-sectional area blood flows through a vascular stent. A conventional stent is a short, wire-mesh tube that acts like a scaffold to help keep the artery open. However, in the context of the present invention a stent is a short tube on which the inner surface is nano-structured for the purpose of measuring blood flow through the vessel segment.

The effect of the induced magnetic field of the nanoscale microwave ferrites on blood flow through the microvessel has been examined [Shit and Roy, 2016]. The nonlinear micropolar fluid model was applied to examine the effect of induced magnetic field on blood flow through a constricted channel. The assumption was that the flow is unidirectional and flowing through a narrow channel, where the Reynolds number is less than unity such as in microvessels. Under the low Reynolds number approximation, the analytical expressions for axial velocity, micro-rotation component, axial pressure gradient, axial induced magnetic field, resistance to flow and wall shear stress were obtained. The flow characteristic phenomena were analyzed by taking valid numerical values of the parameters, which are applicable to blood rheology. There was excellent agreement of the model with the analytical solutions of Hartmann [Hartmann, 1937]. The study showed that, the increasing values of the magnetic field strength decreases the axial velocity at the central line of the channel, while the flow is accelerating in the vicinity of the channel wall. The induced magnetic field has an increasing effect on the micro-rotation component, which in turn produces increasing pressure gradient. The electrical response of the microcirculation increases with the increase in the Hartmann number and the stenosis height. They concluded that, the resultant flow predictions were useful for the potential applications in cardiovascular engineering [Shit and Roy, 2016].

Several types of stents have been used for vascular expansion. The first generation of vascular stents was called bare metal stent (BMS). The BMS are still widely in use, however, there has been numerous observations of in-stent re-stenosis (ISR) after the implantation [Alfonso, 2010]. Drug eluting stent (DES), a revolutionary device to address the problem of ISR, was developed by coating a drugs-loaded polymer onto the BMS. Despite the success of DES to eliminate the ISR, long-term safety and efficacy are questioned due to the late stent thrombosis (LST) reported in numerous clinical trials [Ong et al., 2005]. The first generation DES to receive regulatory approval were Cypher® (Cordis, Warren, New Jersey, USA) and Taxus® (Boston Scientific, Natick, Massachusetts, USA). Cypher consists of 316L SS platform and two permanent polymer coatings of poly(ethelene-co-vinyl acetate) and poly(n-butyl methacrylate), which are the carrier of sirolimus [Wolf et al., 2008]. The Taxus device also applies a 316L SS substrate and a single polymer/drug mixture layer in which poly(styrene-b-isobutylene-b-styrene) coating combined with 1 µg/mm² paclitaxel are adopted [Ranade et al., 2004]. Both of these two stents are based on an appropriate combination of metallic platform, permanent polymer and an anti-proliferative drug. It has been demonstrated that the polymer coating caused persistent arterial wall inflammation and the drug delayed the vascular healing. The ongoing developments of DES concern the core and the coating of the stent as well as the eluted drugs [Daemen and Serruys, 2007]. U.S. Pat. 11,207,448, 2021 to Sasaki et al., describes the biodegradable stents (BDS). Others have developed endothelial progenitor cell (EPC) capture stents [Aoki et al., 2005]. Some of these stents are currently in clinical trials and the outcomes of the studies are highly expected.

The pathogenesis of ISR involves the presence of vascular injury and foreign materials that lead to a disorder of coagulation as well as inflammatory and complement systems. The cascade of events activate neutrophils and macrophages together with the released cytokines and growth factors which accelerate the hyperplasia of smooth muscle cells (SMCs), leading to remodeling of the extracellular matrix (ECM) and initiating smooth muscle cell migration [Welt and Rogers, 2002; Newby and Zaltsman, 2000]. The pathophysiologic end-points are the formation of thrombus and ISR, which are the key points for consideration when novel vascular stents are designed and developed. Currently, based on the BMS, numerous surface modification approaches and novel concept vascular stents aiming at modulating biological responses and improving the stent performance are developed. Attempt have been made to inhibit ISR, using some radioactive stents [Albiero et al., 2000; Leon et al., 2001] and drug eluting stent [Stone et al., 2004; Tung et al., 2006].

The U.S. Pat. 11,207,448 of 2021 to Sasaki et al., described bioasborbable stent made from biodegradable polymer coating stent effective in delaying the damage of physical properties (particularly radial force) of a core structure. The vascular stent includes a core structure of a bioabsorbable material (e.g., Mg), a first coating layer of a first polymer with biodegradability, and a second coating layer of a second polymer with biodegradability, wherein the first coating layer covers the whole surface of the core structure; the second coating layer covers a part or the whole surface of the first coating layer. Furthermore, biodegradable vascular stents can be made of both polymers (lactic acid, glycolic and caprolactone families) and metallic alloys (Mg-based or Fe-based alloys) [Bourantas et al., 2012]. Current biodegradable stents are made from polymer comprising poly (L-lactic acid) (PLLA), which is metabolized into lactic acid, carbon dioxide and water ultimately [Nair et al., 2007]. Some metals like pure magnesium, iron and their alloys have been applied in the design of vascular stents because of their mechanical properties and low toxicity [Moravej et al., 2011].

The metals used in cardiovascular stents could be treated and specially coated to form biocompatible solely titanium oxide layers that have nanoscale microwave ferrites. Junkar et al, (2021) in U.S. Pat. 11,208,720 described a method for treatment of medical devices made from nickel titanium (NiTi) alloys. This enables nano-structuring of the surface of the stent with improved biocompatibility. The platelets do not readily attach and activate on such surfaces and the thrombus formation rate is reduced in comparison with extensively used untreated NiTi alloys.

Recently, investigators have developed nanomaterial designs and integration strategies for the bioresorbable electronic stent with drug-infused functionalized nanoparticles to enable flow sensing, temperature monitoring, data storage, wireless power/data transmission, inflammation suppression, localized drug delivery, and hyperthermia therapy [Son et al, 2015]. In normal persons where no vascular expansion is required, a stent may not be used as the support, rather an adhesive matrix could be used to hold a nano-structured layer of the AI-NANOFLOWMETER. The layer of AI-NANOFLOWMETER could be placed on an adhesive matrix on the surface of the vessel. Such adhesive materials have been developed and used for sealing broken arteries. The latter comprises a biomacromolecule-based matrix hydrogel which can undergo rapid gelling and fixation to adhere and seal bleeding arteries and cardiac walls after UV light irradiation [Hong et al., 2019]. It was demonstrated that, these repairs can withstand up to 290 mm Hg blood pressure, significantly higher than blood pressures in most clinical settings (systolic BP 60-160 mm Hg).

### BRIEF SUMMARY OF THE INVENTION

Currently, there is no unified approach to monitoring of mental performance in real-time. Electrophysiological techniques offer good temporal resolution but have poor sensitivity and specificity in the definition of unified parameters that characterize good versus bad mental performance. On the other hand, neuroimaging techniques (PET and fMRI) are too cumbersome and have poor temporal resolution not suitable for real-time applications for monitoring mental performance. Njemanze (2002) in U.S. Pat. 6,390,979 described a non-invasive transcranial Doppler ultrasound technique for monitoring mental performance based on measurement of cerebral blood flow velocity within the small area of the main stem of the MCA with the pulsed wave Doppler ultrasound sample volume. The method allowed human-machine interface that permits easy and fast computation of subject’s mental performance and communication to a human observer or a host computer. However, the device though portable by comparison to other techniques was not adaptable to many applications for example, in a battlefield situation where mind control of weapon systems could be required. The operation of the system required battery power and hence bulky. The technique could predict overall “neurocognitive” strategies allowing pre-test classification of subjects and prediction of expected results with greater specificity and sensitivity. The neurocognitive strategy at each level of task has some specificity for each individual depending on the approach to problem solving and was characterized as the mental signature. Even though, the Doppler ultrasound device was comparatively portable it required a carry-on device obvious to anyone that such monitoring is on-going. What is required is a method which operates without battery-power, not visible to the eye as it operates at nano-scale level. It could facilitate mental communication with mission control center even in the absence of spoken words. The method could detect changes in cerebral blood flow during mental activity, by conversion to frequency variations and transmission using microwave frequency to a human-machine interface or mission control center.

### OBJECTIVES AND FEATURES OF THE INVENTION

It is a feature of the present invention to provide a system for measurement of cerebral blood flow through a stent or matrix for monitoring mental performance.

It is a feature of the present invention to provide a system for measurement of blood flow through a stent or matrix to determine in-stent re-stenosis of the vessel.

It is a feature of the present invention to provide a system for measurement of cerebral blood flow through a stent for monitoring tasks of general intelligence.

It is a feature of the present invention to provide a system for measurement of cerebral blood flow through a stent to detect the correct response distinct from the false response to a task presented to a subject.

It is a feature of the present invention to provide a system for measurement of cerebral blood flow through a stent for real-time monitoring of mental performance as vital signs of the mental state-of-being of the subject.

It is a feature of the present invention to provide a system for measurement of blood flow through a stent for detection of microembolic signals in the vessel.

It is a feature of the present invention to provide a system for measurement of cerebral blood flow through a stent for diagnosis of cerebral ischemia in patients.

It is a feature of the present invention to provide a system for measurement of cerebral blood flow through a stent for detection of impending syncope.

It is a feature of the present invention to provide a system for measurement of cerebral blood flow through a stent for detection of impending seizures.

It is a feature of the present invention to provide a system for measurement of cerebral blood flow through a stent for monitoring working memory and to communicate the information through a human-machine interface for control of the machine or weapon system.

It is a feature of the present invention to provide a system for measurement of cerebral blood flow through a stent for monitoring working memory in order to communicate with AI computer for regulation of the autonomy decision-making level in a network system.

It is a feature of the present invention to provide a system for measurement of cerebral blood flow through a stent for diagnosis of impaired working memory in subjects or patients with memory deficits.

It is a feature of the present invention to provide a system for measurement of cerebral blood flow through a stent for monitoring of working memory for control of machines through a human-machine interface.

It is a feature of the present invention to provide a system for measurement of cerebral blood flow through a stent for monitoring of working memory for control of the functions of robotic limbs.

It is a feature of the present invention to provide a system for measurement of cerebral blood flow through a stent for monitoring working memory for control of functions of weapon systems.

It is a feature of the present invention to provide a system for measurement of coronary artery blood flow through a stent to the heart to detect the reduction in blood flow due to in-stent re-stenosis or thromboembolism of the vessel.

It is a feature of the present invention to provide a system for measurement of arterial blood flow through a stent to the extremities to detect reduction in blood flow due to in-stent re-stenosis or thrombosis of the vessel.

It is a feature of the present invention to provide a system for measurement of blood flow through a stent to an organ to detect reduction in blood flow due thrombosis of the vessel and for activation of an implanted drug delivery pump for thrombolysis.

It is a feature of the present invention to provide a system for measurement of cerebral blood flow through a stent for monitoring working memory for control of autonomous cars.

It is a feature of the present invention to provide a system for measurement of cerebral blood flow through a stent for monitoring working memory to control functions of construction machines at a remote site.

It is a feature of the present invention to provide a system for measurement of cerebral blood flow through a stent for monitoring working memory for control of functions of robotic devices in Space.

It is a feature of the present invention to provide a system for measurement of cerebral blood flow through a stent for monitoring working memory for control of functions of instruments of telemedicine.

It is a feature of the present invention to provide a system for measurement of cerebral blood flow through a stent for monitoring working memory for control of functions of tele-surgical devices.

It is a feature of the present invention to provide a system for measurement of cerebral blood flow through a stent for monitoring improvements of working memory due to interventional procedures.

It is a feature of the present invention to provide a system for measurement of cerebral blood flow through a stent for monitoring improvements of working memory due to pharmacological interventions.

It is a feature of the present invention to provide a system for measurement of cerebral blood flow through a stent for monitoring the physiologic biomarkers for skills acquisition and automation during mental performance of a task.

It is a feature of the present invention to provide a system for measurement of cerebral blood flow through a stent for monitoring the critical limits of neurocognitive workload of a subject.

It is a feature of the present invention to provide a system for measurement of cerebral blood flow through a stent to the cerebral hemispheres (right, left or both) to determine the effectiveness of task performance, and to explain factors contributing to decrements in performance.

It is a feature of the present invention to provide a system for measurement of cerebral blood flow through a stent for monitoring the effect of perceptual tasks on mental performance.

It is a feature of the present invention to provide a system for measurement of cerebral blood flow through a stent for monitoring the effect of odors on mental state-of-being.

It is a feature of the present invention to provide a system for measurement of cerebral blood flow through a stent for monitoring odor-recognition in the brain of a canine.

It is a feature of the present invention to provide a system for measurement of cerebral blood flow through a stent for monitoring the effect of facial recognition on mental state-of-being.

It is a feature of the present invention to provide a system for measurement of cerebral blood flow through a stent for monitoring the effect of motor tasks on mental performance.

It is a feature of the present invention to provide a system for measurement of cerebral blood flow through a stent for monitoring the effectiveness of countermeasures of extreme environments (microgravity, high-altitude, high temperature, extreme-cold and deep sea diving) on mental performance.

It is a feature of the present invention to provide a system for measurement of cerebral blood flow through a stent for monitoring the effect of hypergravity and the countermeasures such as the G-suit on mental performance.

It is a feature of the present invention to provide a system for measurement of cerebral blood flow through a stent for monitoring the effect of Space suit for extravehicular activity on the human brain.

It is a feature of the present invention to provide a system for measurement of cerebral blood flow through a stent for monitoring mental performance in education, advertisement, politics, industry and entertainment.

It is a feature of the present invention to provide a system for measurement of cerebral blood flow through a stent for monitoring the effects of videogames on mental performance.

It is a feature of the present invention to provide a system for measurement of cerebral blood flow through a stent for monitoring the effects of virtual reality simulations on mental performance.

It is a feature of the present invention to provide a system for measurement of cerebral blood flow through a stent for monitoring the effect of a task (advertorial design of materials, music, automobile, houses, designer clothes) presented in a virtual environment generator on mental performance.

It is a feature of the present invention to provide a system for measurement of cerebral blood flow through a stent for monitoring the effect of a task (advertorial design of speeches including for political campaigns and forensics) presented in a virtual environment generator on mental performance.

Yet another feature of the present invention is to provide a system for measurement of cerebral blood flow through a stent to monitor the effect of motor skill learning after a stroke or neurosurgical operation on mental performance.

Yet another feature of the present invention is to provide a system for measurement of cerebral blood flow through a stent to monitor the effect of a visual linguistic task on mental performance of aphasic patients after a stroke.

Yet another feature of the present invention is to provide a system for measurement of cerebral blood flow through a stent that synchronizes with other implanted devices such as cardiac pacemaker or cardioverter defibrillator in a patient.

Yet another feature of the present invention is to provide a system for measurement of cerebral blood flow through a stent that could synchronize with other implantable stimulator devices such as spinal cord stimulator in a patient.

A further feature of the present invention is to provide a system for measurement of cerebral blood flow through a stent for monitoring the effect of visual non-linguistic task on mental performance of healthy subjects, stroke patients, patients with depression and dyslexia.

It is a feature of the present invention to provide a system for measurement of cerebral blood flow through a stent with nano-scale structured surface for monitoring mental performance in a subject comprising human being and canine.

It is a feature of the present invention to provide a system for measurement of cerebral blood flow through a biodegradable stent with nano-scale structured surface for monitoring mental performance in a subject.

It is a feature of the present invention to provide a system for measurement of cerebral blood flow through a stent for monitoring mental performance in a large population of people at a very affordable price for use in the workplace, school, home or battlefield.

It is a feature of the present invention to provide a system for measurement of cerebral blood flow through a stent for monitoring mental performance in users with no special operational skills.

It is a feature of the present invention to provide a system for measurement of cerebral blood flow through a stent for monitoring mental performance repeatedly non-invasively for several hours throughout the day.

It is a feature of the present invention to provide a system for measurement of cerebral blood flow through a stent for monitoring mental state-of-being during sleep for several hours.

It is a feature of the present invention to provide a system for measurement of cerebral blood flow through a stent in real-time with high temporal resolution for monitoring of mental performance.

It is a feature of the present invention to provide a system for measurement of cerebral blood flow through a stent for real-time monitoring of mental performance of several subjects simultaneously connected to a computer network.

It is a feature of the present invention to provide a system for measurement of cerebral blood flow through a stent for monitoring the mental performance of workers to maximize productivity.

It is a feature of the present invention to provide a system for measurement of cerebral blood flow through a stent for monitoring mental performance of workers in a production system for optimizing the time motion studies.

It is a feature of the present invention to provide a system for measurement of cerebral blood flow through a stent for monitoring mental performance for use in organization of work schedule and programs.

It is a feature of the present invention to provide a system for measurement of cerebral blood flow through a stent for monitoring the mental state-of-being in relation to the circadian biorhythm of the individual.

It is a feature of the present invention to provide a system for measurement of cerebral blood flow through a stent to monitor mental state-of-being during the hormonal fertility cycle for improving fertility and natural family planning such as the Creighton Model (CrM), a natural or fertility awareness based method of family planning based on a woman’s observations of her cervical fluid or mucus.

It is a feature of the present invention to provide a system for measurement of cerebral blood flow through a stent for monitoring mental performance of subjects on a mission.

It is a feature of the present invention to provide a system for measurement of cerebral blood flow through a stent for monitoring of mental performance of a subject and communicate same via a cellular phone including 5G network to a host computer.

It is a feature of the present invention to provide a system for measurement of cerebral blood flow through a stent to obtain mental signature for cognitive biometric information for access to high security networks comprising avionic systems, nuclear plant, ammunition network, air traffic control.

It is a feature of the present invention to provide a system for measurement of cerebral blood flow through a stent to obtain the mental signature for forensic crime prevention that may indicate that a criminal subject under probation maybe about to commit a crime.

It is a feature of the present invention to provide a system for measurement of cerebral blood flow through a stent to obtain mental signature of a subject for access to computer network security for financial transactions with digital currency such as bitcoin.

It is a feature of the present invention to provide a system for measurement of cerebral blood flow through a stent for monitoring mental performance that translates into movement of artificial hands and/or legs in a subject with limb amputation.

It is a feature of the present invention to provide a system for measurement of cerebral blood flow through a stent to obtain mental signature of a subject for use as cognitive biometric identity on a high military security network.

It is a feature of the present invention to provide a system for measurement of cerebral blood flow through a stent for monitoring mental state-of-being of a subject for access to high-security network.

It is a feature of the present invention to provide a system for measurement of cerebral blood flow through a stent for monitoring mental performance of a subject that correlates with speech, computer-aided speech for patients with speech impairment.

It is a feature of the present invention to provide a system for measurement of cerebral blood flow through a stent for monitoring mental performance of a subject for facial recognition.

It is a feature of the present invention to provide a system for measurement of cerebral blood flow through a stent for monitoring mental performance of a subject who is a criminal released on probation to detect the mental signature denoting an impending motivation to commit a violent crime.

It is a feature of the present invention to provide a system for measurement of cerebral blood flow through a stent for monitoring mental performance of a patient with depression and to assess effectiveness of interventional measures.

It is a feature of the present invention to provide a system for measurement of cerebral blood flow through a stent for monitoring mental performance of psychiatric patient and to assess effectiveness of interventional measures.

It is a feature of the present invention to provide a system for measurement of cerebral blood flow through a stent for detection of aura of seizures in patients.

It is a feature of the present invention to provide a system for measurement of cerebral blood flow through a stent for monitoring mental performance of a subject that is stored in blockchain.

It is a feature of the present invention to provide a system for measurement of cerebral blood flow through a stent for monitoring mental performance of subjects connected to neural network of artificial intelligent (AI) system.

It is a feature of the present invention to be used at seaport and airports in a canine attached with GPS unit and implanted with the device of the present invention to detect changes in cerebral blood flow during perception of a target odor such as TNT explosives.

It is a feature of the present invention to be used for biometric assessment of visitors by an immigration officer implanted with device of the present invention to monitor cerebral blood flow changes during perception of target (eg terrorist) face, to trigger an alert to other security personnel at the border.

### BRIEF DESCRIPTION OF SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1 shows the block diagram of one form of embodiment of the present invention.

FIG. 2 shows the block diagram of another modification of the embodiment of the present invention.

FIG. 3 shows a schematic microscopic surface on the vascular stent according to the present invention.

FIG. 4 shows a schematic microscopic surface on the vascular stent during placement in the vessel on a catheter according to the present invention.

FIG. 5 shows a schematic microscopic surface on the vascular stent put in place in the vessel according to the present invention.

FIG. 6 shows the arteries of the brain blood circulation.

FIG. 7 shows the arteries of the Circle of Willis that supply blood to the major brain areas.

FIG. 8 shows the stent with the inner surface of the vascular stent deployed in an artery of the Circle of Willis to monitor cerebral blood flow according to the present invention.

FIG. 9 shows one type of activation pattern (black polygons) of the microscopic nano-structure of microwave ferrites on the surface layer of the present invention.

FIG. 10 shows another type of activation pattern (black polygons) of the microscopic nano-structure of microwave ferrites arranged on the surface layer of the present invention.

FIG. 11 shows yet another type of activation pattern (black polygons) of the microscopic nano-structure of microwave ferrites arranged on the surface layer of the present invention.

FIG. 12 shows one embodiment of the present invention affixed to a vessel in the brain and the transmission and reception of microwave signals.

FIG. 13 shows the flow chart of function of the computer program of the present invention, illustrated by way of example.

### DETAILED DESCRIPTION OF THE INVENTION

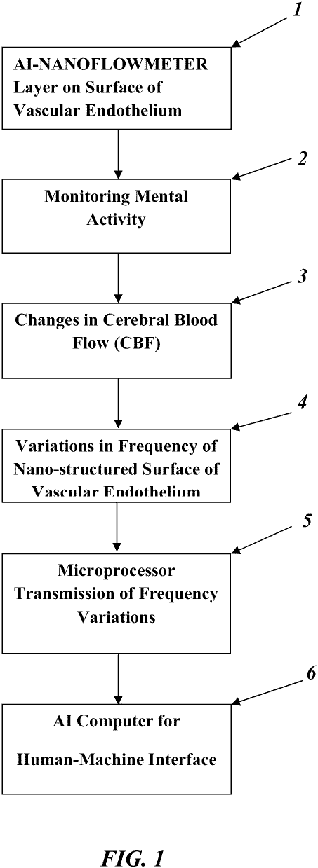

FIG. 1 shows the block diagram of one form of embodiment of the present invention. The AI-NANOFLOWMETER layer is placed on the surface of vascular endothelium 1, for the purpose of monitoring mental activity 2, which correlates with changes in cerebral blood flow (CBF) 3. The changes in CBF induce variations in frequency of the nano-structured surface of the stent 4. A microprocessor processes and transmits the frequency variations 5, to an AI computer for human-machine interface 6. The microprocessor could be programmed to apply spectral analysis including using Fast Fourier Analysis for the processing of the frequency variations. The CBF spectrum could be displayed on a device operatively connected to receive microwave signals from the microprocessor such as a cell phone. The human-machine-interface could facilitate input into the function of machines such as computers, medical devices and weapons systems; for example, a cardiac pacemaker could be synchronized to cerebral blood flow measurements using the present invention.

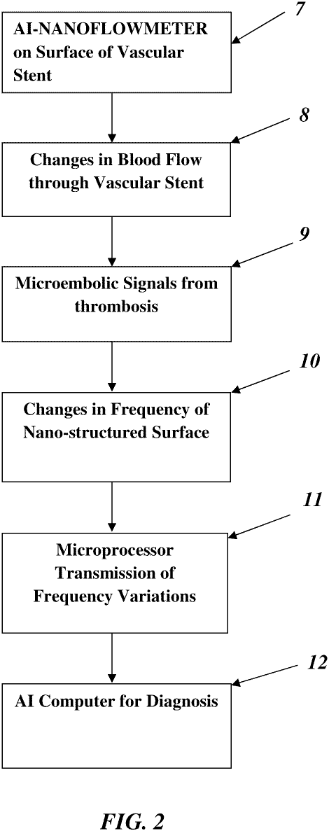

FIG. 2 shows the block diagram of another modification of the embodiment of the present invention. The AI-NANOFLOWMETER layer is placed on the surface of vascular stent 7, for the purpose of monitoring CBF through the stent 8, as well as detect microembolic signals 9, arising from thromboembolism of the vessel orifice of the stent. The changes in CBF and the presence of microembolic signals induce changes in the frequency composition from the nano-structured surface of the stent 10. The microprocessor processes and transmits the frequency variations 11, to an AI computer for diagnosis of the problem 12.

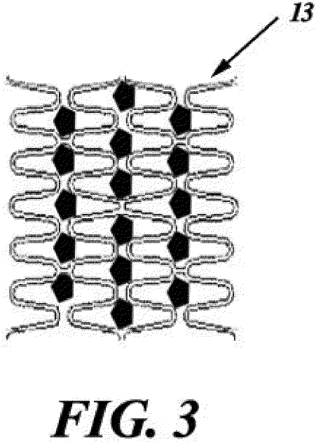

FIG. 3 shows a schematic microscopic surface on the vascular stent according to the present invention. It shows a section of a stent, comprising a small, metal mesh tube. The section of the strut of the stent 13, has on and in-between the scaffold the inner cover layer of black polygons comprising nano-scale microwave ferrites. The polygons cover the entire inner surface of the stent. The stent is inserted into a blood vessel in a collapsed state on a catheter. The stent could be self-expanding, that is, sheathed in retractable delivery system and spontaneously expands. The stent could be mounted on an angioplasty balloon called balloon-mounted, which could be inflated to deploy. The surface of the stent could be covered as well with medication and is called drug-eluting stent. The stent could be made from biodegradable polymer, metal alloy with drug coating.

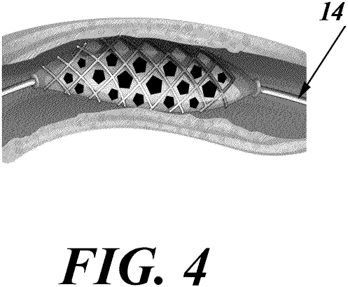

FIG. 4 shows a schematic microscopic surface on the vascular stent during placement in the vessel on a catheter according to the present invention. It shows a catheter and balloon 14 for delivery of the stent with its surface covered with black polygons of nano-scale microwave ferrites. The delivery of the stent is facilitated by a catheter and balloon 14. The balloons and stents come in different sizes to match the size of the diseased artery. They are expanded inside the vessel to prop the walls open in the case of narrowing. The inner surface is covered by polygons of nano-scale microwave ferrites as shown. The guide wire of the catheter delivery system and balloon 14 are used to place the stent in the desired position. The procedure for placement of the stent is similar to that described for vascular angioplasty for endovascular intervention [Silva et al. 1996]. In angioplasty, a balloon-tipped catheter is used to open a blocked blood vessel and improve blood flow. In the present invention, we apply medical imaging, typically live x-rays, to guide the catheter to the desired position of the stent, then the balloon is inflated to attach the stent to the vessel wall and allow blood flow through. The stent is left inside the blood vessel, in case the vessel was narrow at the point of placement, it will help keep it open. Just like in angioplasty, the placement of the stent is minimally invasive and usually does not require general anesthesia or overnight stay in the hospital.

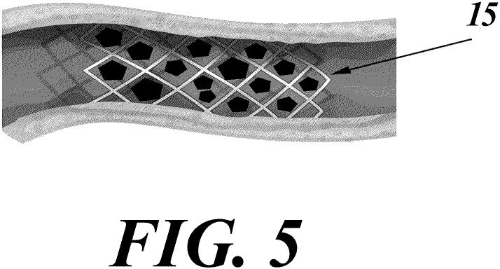

FIG. 5 shows a schematic microscopic surface on the vascular stent put in place in the vessel according to the present invention. It shows the stent in place expanded inside the vessel. The stent inner surface layer 15 has on and in-between the scaffold the nano-scale microwave ferrites. The nano-structured layer is the inner surface of the stent in direct contact with flowing blood.

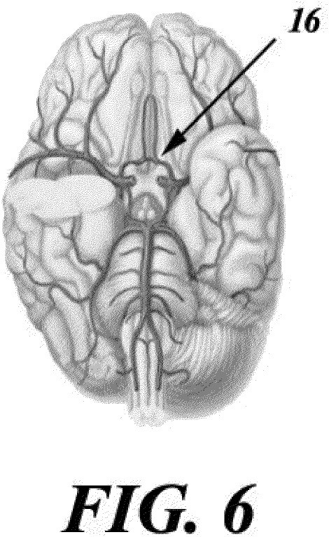

FIG. 6 shows the arteries of the brain blood circulation. It shows the location of the arteries of the brain at the base of the skull 16 into which the stent could be placed.

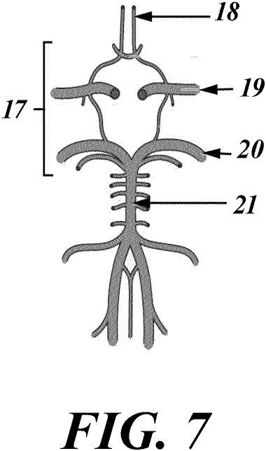

FIG. 7 shows the arteries of the Circle of Willis that supply blood to the major brain areas. It shows the arteries of the Circle of Willis 17 that supply blood to the major brain areas, comprising the right anterior cerebral artery (RACA) 18, the right middle cerebral artery (RMCA) 19, right posterior cerebral artery (RPCA) 20, and basilar artery (BA) 21. The stent could be implanted in one artery on the right, left or both sides depending on the indication for use.

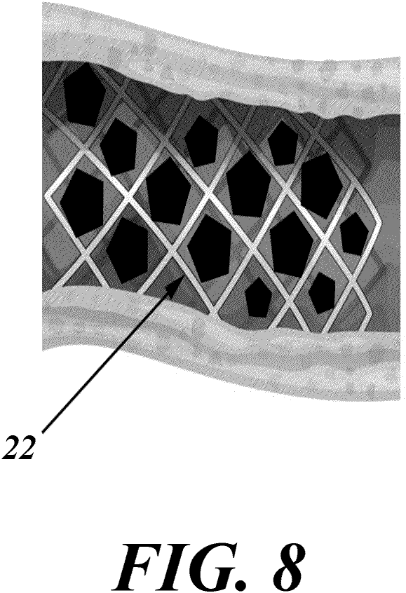

FIG. 8 shows the stent with the inner surface of the vascular stent deployed in an artery of the Circle of Willis to monitor cerebral blood flow according to the present invention. It shows the mesh of stent 22 expanded inside the vessel and the inner surface covered by the nano-scale microwave ferrites shown as black polygons in direct contact with the flowing blood.

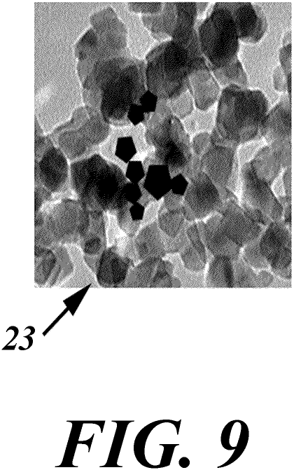

FIG. 9 shows one type of activation pattern (black polygons) of the microscopic nano-structure of microwave ferrites on the surface layer of the present invention. The microwave ferrites at the centre 23 represented by black polygons are activated by blood flow projectile at the center.

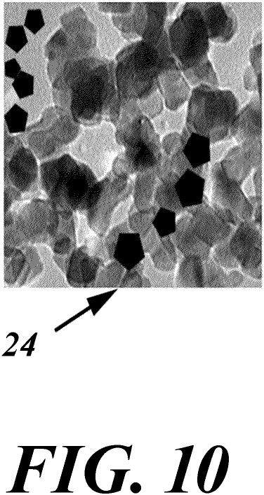

FIG. 10 shows another type of activation pattern (black polygons) of the microscopic nano-structure of microwave ferrites arranged on the surface layer of the present invention. The microwave ferrites (black polygons) closer to the vessel walls 24 are activated by boundary layer flow conditions at the walls.

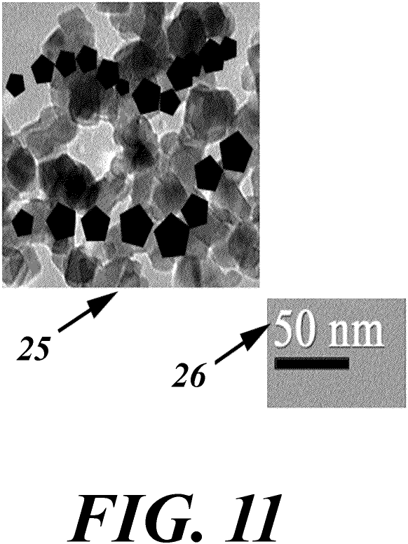

FIG. 11 shows yet another type of activation pattern (black polygons) of the microscopic nano-structure of microwave ferrites arranged on the surface layer of the present invention. The microwave ferrites (black polygons) closer to the walls 25 of the stent are activated by flow at the boundary layer, while those at the center have been activated by the central projectile. The microwave ferrites are usually less than 50 nm in size 26.

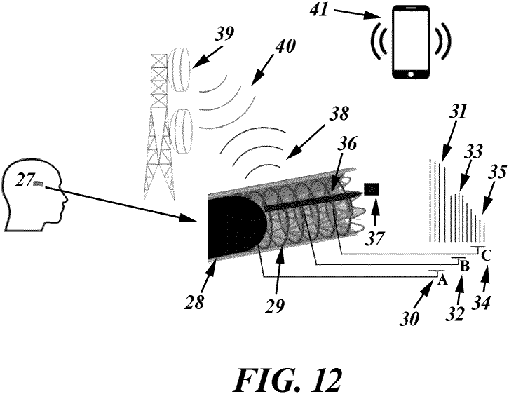

FIG. 12 shows one embodiment of the present invention affixed to a vessel in the brain and the transmission and reception of microwave signals. A stent 27 with the inside surface layer made according to the teachings of the present invention and implanted in the brain artery. The cerebral blood flow through the arterial stent 28 induces changes in frequency of the microwave ferrites proportional to velocity 29, which varies according to the location of the ferrites, with frequency from the center projectile 30, highest in systole 31, lower at the near-wall 32 at the beginning of diastole 33, and at the wall in end-diastole 34, the frequency is least 35. The frequency changes sum up across the stent material 36, and are processed by the microprocessor 37 for onward transmission 38. The processing may include spectral analysis of the frequency signals that could be processed with a spectrum analyzer. The microwave antennae 39 could also re-transmit 40 the information to a microwave receiver such as a cell phone 41, host computer or weapon system.

FIG. 13 shows the flow chart of function of the computer program of the present invention, illustrated by way of example. The system starts 42, monitoring the CBF in both MCAs during baseline activity 43. The baseline measured CBF values are stored 44. The system monitors CBF during the study mental activity 45, which is compared to the baseline data 46. If the values of CBF are within the set limits 47, then it continues the monitoring of CBF. However, if the values of CBF are not within the set limits, it proceeds to check for artifacts. If artifacts are present 48, it continues to monitor CBF, however, if not present, the system compares CBF on the right and left sides of the brain 49, by calculating the laterality index (LI) 50:

$$\text{LI′ =} \left({\text{CBF}}_{\text{RMCA}} - {\text{CBF}}_{\text{LMCA}}\right) / \left({\text{CBF}}_{\text{RMCA}} + {\text{CBF}}_{\text{LMCA}}\right) * 100.$$

The actual magnitude of lateralization (LI) for each time interval for each paradigm is calculated as the difference between LI′ values measured during the time of the task and the corresponding time segment at baseline:

$$\text{LI} = {\text{LI′}}_{\text{task}} - {\text{LI′}}_{\text{baseline}}$$

In general, positive LI values suggest right lateralization, while negative LI values suggest left lateralization. Zero LI values showed no lateralization from the baseline condition or possible bilateral response. If the subject is a man, then right hemisphere lateralization could be presumed to be for intelligent decision, while in women, a left hemisphere lateralization would be presumed to be for the intelligent decision 51. The information is transmitted to the AI computer 52 for applications of the AI algorithm, for example, for regulation of autonomy decision-making level in the network.

We claim:

1. A system for cerebral blood flow measurement comprising: a flow sensor layer on a vascular stent or matrix placed inside a cerebral vessel, wherein the said flow sensor is for monitoring mental performance of a subject.

2. The system for cerebral blood flow measurement as in claim 1, wherein the said flow sensor layer further comprising a nano-structured layer of microwave ferrites.

3. The system for cerebral blood flow measurement as in claim 1, wherein the said flow sensor measured mental performance is the mental state-of-being of the subject.

4. The system for cerebral blood flow measurement as in claim 1, wherein the said flow sensor measured mental performance determines the neurocognitive strategy for intelligent decision-making.

5. The system for cerebral blood flow measurement as in claim 1, wherein the said flow sensor monitors cerebral blood flow in conditions comprising cerebral ischemia, sleep, syncope, effects of positive Gz-acceleration and seizures.

6. The system for cerebral blood flow measurement as in claim 1, wherein the said flow sensor measures mental performance simultaneously in a number of subjects on a computer network.

7. The system for cerebral blood flow measurement as in claim 1, wherein the said flow sensor measures mental performance used to control computers, machines and weapon systems, by communication through human-machine interface comprising wireless cellular phone network.

8. The system for cerebral blood flow measurement as in claim 1, wherein the said flow sensor measures mental performance and communicates with the AI computer program for regulation of autonomy decision-making level in a network comprising avionic computer system, high-security network and digital financial transaction network.

9. The system for cerebral blood flow measurement as in claim 1, wherein the said flow sensor measures mental performance as the working memory in a patient with diseases comprising neurodegenerative disease, stroke, depression, and psychiatric disorders.

10. The system for cerebral blood flow measurement as in claim 1, wherein the said flow senor measures mental performance used to control function of machinery comprising robotic limbs, artificial limbs, construction machines, and tele-medicine equipment.

11. A system for cerebral blood flow measurement comprising: a flow sensor layer of nano-structured microwave ferrites on a biodegradable vascular stent placed inside the cerebral artery; signals from said microwave ferrites are processed and transmitted by a microprocessor for monitoring mental performance of a subject.

12. The system for cerebral blood flow measurement as in claim 11, wherein the said flow sensor monitors mental performance for control of devices comprising driver-less car, tele-surgery, construction equipment, anti-gravitational suit and extravehicular activity suit.

13. The system for cerebral blood flow measurement as in claim 11, wherein the said flow sensor measures mental performance used as mental signature during processing of stimuli comprising facial, color, odor, linguistic, non-linguistic stimuli, cognitive biometric stimuli and forensic stimulus analysis.

14. The system for cerebral blood flow measurement as in claim 11, wherein the said flow sensor measures mental performance synchronized with function of other devices comprising cardiac pacemaker, implantable cardioverter defibrillator, implantable drug delivery system, electroencephalograph, Doppler ultrasound, laser Doppler, brain electrical potential, and pain stimulator devices.

15. The system for cerebral blood flow measurement as in claim 11, wherein the said flow sensor measures mental performance used to determine changes in the hormonal fertility cycle.

16. The system for cerebral blood flow measurement as in claim 11, wherein the said flow sensor measures mental performance used as mental signature for access to high security computer network comprising digital financial transactions, air-traffic control, nuclear plant, ammunition and advanced military weapons and avionic systems.

17. The system for cerebral blood flow measurement as in claim 11, wherein the said flow sensor measures mental performance used to control activities comprising speech production, computer-aided speech and other speech impairments in subjects.

18. The system for cerebral blood flow measurement as in claim 11, wherein the said flow sensor measures mental performance for diagnosis and treatment of diseases comprising depression, sleep abnormality, autism, dyslexia, schizophrenia, stroke, dementia and other neurodegenerative diseases.

19. A system for blood flow measurement comprising: a flow sensor layer of nano-structured layer of a vascular stent or matrix inside a vessel, said flow sensor detects micro-embolic signals passing through the vessel for effective thrombolysis.

20. The system for blood flow measurement as in claim 19, wherein the said flow sensor detects in-stent re-stenosis of the vessel.
